(19) United States
(12) Patent Application Publication
(10) Pub. No.: US 2006/0084172 A1
(43) Pub. Date: Apr. 20, 2006

(54) Method for producing stem cells with increased developmental potential

(75) Inventors: Albrecht Muller, Wurzburg (DE); Nicole Kirchhof, Wurzburg (DE); Friedrich Harder, Wurzburg (DE)

Correspondence Address: MAYER, FORTKORT & WILLIAMS, PC, 251 NORTH AVENUE WEST, 2ND FLOOR, WESTFIELD, NJ, 07090, US

(73) Assignee: Julius-Maximilians-Unversitat Wurzburg, Wurzburg (DE)

(21) Appl. No.: 10/498,475

(22) PCT Filed: Nov. 29, 2002
(86) PCT No.: PCT/DE02/04459

§ 371 Date: May 16, 2005

(30) Foreign Application Priority Data
Dec. 10, 2001 (DE) 101 62 080.2

(51) Int. Cl. C12N 15/87; C12N 5/08

(52) U.S. Cl. 435/455; 435/368; 435/372

(57) ABSTRACT

The invention relates to a method for producing stem cells having an increased development potential from somatic stem cells, wherein a tissue sample comprising somatic stem cells or a body fluid sample comprising somatic stem cells is taken from an organism, wherein from this tissue sample or body fluid sample as an option somatic stem cells are isolated and/or cultivated, and wherein the thus obtained somatic stem cells are treated with a substance modulating the methylation of the DNA of the cells or a substance modulating the acetylation of chromatin of the cells.

### FIELD OF THE INVENTION

The invention relates to a method for producing stem cells having an increased development potential, wherein a tissue sample is taken from an organism, and wherein from this tissue sample stem cells are isolated and as an option cultivated. The invention further relates to somatic stem cells (e.g. neural, hematopoietic, mesenchymal, epithelial stem cells), which can be produced in this way, as well as to various uses of such stem cells.

### BACKGROUND OF THE INVENTION AND PRIOR ART

During the embryonic and fetal development of an organism, stem cells constitute by differentiation to specialized effector cells the developing organism. The totipotent and/or pluripotent cells of the early embryo have a wide development potential, which according to previous findings has to a high extent been lost for the somatic stem cells in the adult tissues. The somatic stem cells develop and maintain a multitude of in part highly specialized cell types and secure the homeostasis of many tissues and organs.

The capability of embryonic totipotent or pluripotent stem cells to differentiate to all tissues or organs has raised hopes with regard to a (partial) replacement of organs or a (partial) repair of organs. However, in particular the production of embryonic stem cells is a cause for very severe ethical concerns. For this reason, somatic cells have to a higher degree become the subject matter of scientific investigations. Most recent findings show that somatic stem cells can also have a substantial development capacity. Neural stem cells can develop blood cells, whereas blood stem cells can produce brain and muscle cells in vivo. With regard to this transdifferentiation, reference is for instance made to the documents U.S. Pat. No. 6,087,168 and U.S. Pat. No. 6,093,531. Scientific publications in the last years, too, show that a number of somatic stem cells have a higher development potential than assumed up to now [see Wei G. Schubiger G, Harder F, Müller A M (2000) Stem cell plasticity in mammals and transdetermination in Drosophila; common themes? Stem Cells, 18:409-414]. On the one hand, plasticity could be observed within a stem cell system, for instance reactivation of embryonic gene expression patterns for the transplantation of somatic hematopoietic stem cells in early mouse embryos. On the other hand, the generation of heterologous stem cells after the transplantation of highly enriched stem cells was described [see Wei G, Schubiger G, Harder F, Müller A M (2000) Stem cell plasticity in mammals and transdetermination in Drosophila; common themes? Stem Cells, 18:409-414 and Geiger H, Sick S, Bonifer C, Müller A M (1998) Globin gene expression is reprogrammed in chimeras generated by injecting adult hematopoietic stem cells into mouse blastocysts, Cell. 93:1055-1065]. Neural stem cells obtained from the brain of mice could settle in the blood system of irradiated receiver animals, even after in vitro culture for months, and generate myelo-erythroid as well as lymphoid cells [see Bjornson C R R, Rietze R L, Reynolds B A, Magli M C, Vescovi A L (1999) Turning brain into blood: a hematopoietic fate adopted by adult neural stem cells in vivo, Science 283:534-537]. In addition to an ectodermic to mesodermic transformation of neural stem cells of the mouse, human and murine neural stem cells could also generate muscle cells in vitro. Another example for the plasticity of adult stem cells are hematopoietic stem cells, which participated in the liver regeneration as well as the generation of microglial and macroglial cells in the brain of adult mice [see Lagasse E, Connors H, Al-Dhalimy M, Reitsma M, Dohse M, Osborne L, Wang X, Finegold M, Weissman I L, Grompe M (2000) Purified hematopoietic stem cells can differentiate into hepatocytes in vivo, Nat. Med. 6:1229-1234]. Equally, after the transplantation of HCSs into irradiated mice, descendants of these cells with now neural phenotype could be found [see Mezey E, Chandross K J, Harta G, Maki R A, McKercher S R (2000) Turning blood into brain: cells bearing neuronal antigens generated in vivo from bone marrow, Science 290:1779-1782]. Bone marrow cells seem to have a potentially high therapeutic benefit, also because they generated new myocardium cells after the transplantation into a myocardial infarction model or settled in the liver after the injection and performed there liver-specific biochemical functions [see Orlic D, Kajstura J, Chimenti S, Jakoniuk I, Anderson S M, Li B, Pickel J, McKay R, Nadal-Ginard B, Bodine D M, Leri A, Anversa P (2001) Bone marrow cells regenerate infarcted myocardium, Nature 410:701-705].

The hematopoietic system is the stem cell system, which is best characterized up to now. Hematopoietic stem cells exist at different development stages in various tissues, such as the fetal liver, the umbilical cord blood and the bone marrow [see Bonifer C, Faust N, Geiger H, Müller A M (1998) Development changes in the differentiation capacity of hematopoietic stem cells, Immunology Today 19:236-241]. Of course they are very rare, can however be highly enriched in vitro by means of monoclonal antibodies and can be found in the somatic bone marrow with a density of one cell per 10⁴ to 10⁵ cells. The highly regenerative potential of this stem cells can be estimated by that an injection of 20 to 40 hematopoietic stem cells isolated from the bone marrow of adult mice can repopulate the complete blood system for life [see Kirchhof N, Harder F, Petrovic C, Kreutzfeldt S, Schmittwolf C, Dürr M, Mühl B, Merkel A, Müller A M (2001) Developmental potential of hematopoietic and neural stem cells: unique or all the same? Cells Tissue Organs (in press)].

Neural stem cells can be detected, among other places, in the subventricular zone and in the hippocampus of the adult brain. These neural stem cells can generate on the one hand new stem cells and can differentiate on the other hand to the three main cell types of the central nervous system, astrocytes, oligodendrocytes and neurons. Neural stem cells can, in contrast to hematopoietic stem cells, the effective multiplication of which has been found to be difficult in in vitro culture systems, be multiplied in cell culture. Neural stem cells of the fetal and adult brain can be excited in vitro to proliferation in presence of FGF-2 (fibroblast growth factor 2) and EGF (epidermal growth factor). They form little balls, so-called neurospheres containing the neural stem cells. In the neurospheres, about one out of 26 cells is a neural stem cell [see Kirchhof N, Harder F, Petrovic C, Kreutzfeldt S, Schmittwolf C, Dürr M, Mühl B, Merkel A, Müller A M (2001) Developmental potential of hematopoietic and neural stem cells: unique or all the same? Cells Tissue Organs (in press)]. Neural stem cells growing in vitro are themselves subjected to regeneration divisions and have a differentiation potential for the generation of neuronal, astrocytic and oligodendrocytic cell types [see Okada S, Nakauchi H, Nagayoshi K, Nishikawa S -I, Miura Y, Suda T (1992) In vivo and in vitro stem cell functions of c-kit and Sca-1-positive murine hematopoietic cells, Blood 80:3044-3050].

In spite of the detection that somatic stem cells can in certain cases generate different tissues, investigations have shown that hematopoietic as well as neural stem cells can preferably again be found, after injection in blastocysts, in their original tissue. Hematopoietic stem cells mainly settle in blood organs and produce blood cells, whereas neural stem cells preferably settle in neural tissues and generate the latter. This behavior is found for murine as well as for human hematopoietic stem cells and for murine neural stem cells [see among other documents Harder F, Lamers M C, Henschler R, Müller A M (2001) Human hematopoiesis in murine embryos and adults following the injection of human HSCs into blastocysts, Blood (in press), and Harder F, Kirchhof N, Müller A M (2001) Tissue specific repopulation preferences of somatic stem cells (manuscript in preparation)]. To a lower degree, thus the generation of cells not belonging to the stem cell system of the used stem cells is found. These data support the assumption that somatic stem cells mainly form cells of their tissue and only secondarily cells of other tissues. These results indicate that the generation of foreign cells (plasticity) is a rare event for somatic stem cells and is not the standard behavior.

### TECHNICAL OBJECT OF THE INVENTION

Therefore, the invention is based on the technical object to provide a method for increasing the plasticity of somatic stem cells.

### BASICS OF THE INVENTION AND PREFERRED EMBODIMENTS

For achieving this technical object, the invention teaches a method for producing stem cells having an increased development potential from somatic stem cells, wherein a tissue sample comprising stem cells or a body fluid sample comprising stem cells is taken from a preferably non-fetal organism, wherein from this tissue sample or body fluid sample as an option somatic stem cells are isolated and/or cultivated and/or transformed with a defined foreign nucleic acid, which is under the control of an operatively connected regulatory element, and wherein the thus obtained somatic stem cells are treated with a substance modulating the methylation of the DNA and/or a substance modulating the chromatin acetylation. More generally expressed, the somatic stem cells are treated with a substance or several different substances, which regulate the transcription of the DNA or genes of the DNA being per se inactive up. Hereby, the plasticity of the somatic stem cells compared to the untreated stem cells is increased, i.e. multipotent somatic stem cells are so to speak transformed into stem cells having a comparatively higher development potential. Multipotency is here the development potential of untreated stem cells. Suitable methods for the comparison of the plasticity or development capacity of treated and untreated stem cells can be taken from the examples of execution.

The production method according to the invention is in principle exclusively used in vitro. For the nomination USA applies however that the method can be performed in vitro or in vivo.

The invention is based on investigations of the cell type specificity of somatic stem cells and in this connection of the regulation of the cell identities and of the plasticity on a molecular level. The basic substance of the chromosomes is called chromatin. Chromatin is composed of acid and basic proteins, and in particular the basic histones are important. By means of these proteins, the DNA is brought into a compact form. The proteins act however also as regulators of the gene expression, the activity of which is in turn regulated by modifications. Thus, the gene expression is increased for instance by a hyperacetylation of the histones. The number of acetylations of the chromatin is naturally regulated by the activity of histone acetylases (HAT) and deacetylases (HDAC). Therefore, in a preferred embodiment of the invention, the substance modulating the chromatin acetylation is a substance reducing the chromatin acetylation and is selected from the group comprising “acetylation activators, histone acetylase activators and histone deacetylase inhibitors, and mixtures of such substances”. However, antagonists thereto can also be used. A histone deacetylase inhibitor is for instance Trichostatin A. Other examples for modulators are nucleoplasmin, chlamydocin, HC-toxin, Cyl-2, WF-3161, DMSO, butyrate, e.g. Na-n butyrate, depudecin, radicocol, substances according to WO97/35990, oxamflatin, apidicin, depsipeptides and trapoxin, including similar cyclic tetrapeptides with modified amino acids, such as 2-amino-8-oxo-9,10-epoxy-decanoic acid (see e.g. Closse et al., Helv. Chim. Acta 57:533-545 (1974), Liesch et al., Tetrahedron 38:45-48 (1982), Umehara, Antibiot. 36:478-483 (1983), Kwon et al., Proc. Natl. Acad. Sci. USA 95:3356-3361 (1998), “Histone Deacetylase Inhibitors: Inducers of Differentiation or Apoptosis of Transformed Cells”, Journal of National Cancer Institute, Vol. 92, No. 15, Aug. 2, 2000). Examples for histone acetylase activators are the proteins p300/CBP and pCAF as well as small molecules mimicrying the activity of these endogenous proteins. On the other hand, the gene expression is also regulated by chemical modifications of the genomic DNA. Methylation of the DNA is also a reason for a suppression of the transcription. Hypermethylation, in particular of 5-methylcytosine, in most cases causes a reduction of the gene expression. Methylation is assumedly involved in selective repression mechanisms for certain gases. The degree of methylation of the genome is determined by special enzymes, methylases and demethylases. These can be inhibited or activated. Inhibition is for instance possible by means of methylase-specific antibodies. Further, it is possible, by the incorporation of modified nucleotides, which cannot be methylated, to terminate repression mechanisms, such that transcription can take place to a higher degree (e.g. 5-aza-2′deoxycytidine).

The concentrations of the substance modulating the methylation of the DNA and/or the chromatin acetylation are typically in the range 1 to 5,000 nanomolar, for instance 50 to 1,000 nanomolar.

In the method according to the invention, an incubation with a cytokine or a mixture of different cytokines can be performed before, during or after the treatment with a substance modulating the methylation of the DNA and/or the chromatin acetylation. Examples for cytokines are IL-1, IL-2, IL-3, IL-6, IL-11, IL-12, CSF, LIF. Preferred is a mixture containing IL-3 and IL-6. Other growth factors, such as EGF and FGF-2, can of course also be used.

The invention thus uses the findings that a termination of repression mechanisms in somatic stem cells is accompanied by an increase of the development potential. Hereby finally an increase of the cell formation potential being similar to that of the embryonic stem cells is induced. It is particularly advantageous if no embryonic materials are however needed for the production of stem cells according to the invention. Rather, somatic stem cells anyway occurring in fetal or adult organisms can be taken and treated according to the invention. This has a special importance by that autologous somatic stem cells having an increased differentiation potential can be generated. This means that from a patient to be treated with stem cells according to the invention first stem cells are taken, subjected to the method according to the invention and thereafter administered again to the patient as a pharmaceutical composition. This autologous procedure secures that virtually no undesired immune reactions will happen, as for instance with allogeneic procedures. If only allogeneic stem cells according to the invention are available, the simultaneous application of immune suppressants known from the transplantation medicine may be recommended.

The invention further relates to the use of stem cells according to the invention having an increased differentiation potential for producing a pharmaceutical composition. Here may be involved for instance neural or hematopoietic stem cells or stem cells originating from the epidermis. The applications of stem cells according to the invention are numerous. For instance, they can be used for the treatment of degenerative diseases of the central nervous system (for instance Parkinson's disease), diabetes, diseases with pathologically low blood cell counts, muscular dystrophy, HSC transplantation after high-dose chemotherapy/radiotherapy during cancer therapies, myocardium cell replacement after a heart attack, skin replacement, cartilage replacement, liver regeneration after cirrhosis of liver, metabolic diseases or age-related tissue degeneration.

For the purpose of the invention, various embodiments are possible. It is for instance possible that the somatic stem cells are subjected to a combined treatment with one or several substances modulating the methylation of the DNA on one hand and one or several substances modulating the chromatin acetylation on the other hand. The two above treatment components can be used at the same time or one after the other (in an arbitrary order). An example of such a combined treatment is the treatment with Trichostatin A and with 5-aza-2′ deoxycytidine in a mixture.

Before, during or after the cultivation of the somatic stem cells or the treatment thereof with a substance modulating the methylation of the DNA and/or a substance modulating the chromatin acetylation, these can also be transformed with a foreign nucleic acid, for instance a therapeutically effective nucleic acid and/or a nucleic acid coding for a biologically functionless marker. It is understood that a suitable regulatory element, such as for instance a promoter, is operatively linked to the nucleic acid or the gene. The transformation can be made in a way being usual in this field, for instance by means of viral vectors containing the foreign nucleic acid. As a marker can for instance be used antibiotic resistance genes, such as resistance against G418 or Hygromycin, HSV-tk gene, NeoR, NGFR, GFP, DHFR, hisD, murine CD24, murine CD8a and others. The therapeutic gene can in principle be arbitrary. Genes coding for expression products, which are inhibitors of genes overexpressing for disease reasons, such as for instance in the case of tumor cells, can be used. Specific examples can be found in a large number in the literature. Such inhibitors are for instance antibodies or binding fragments of antibodies. Further are mentioned here, in particular in oncologic situations, genes coding for toxins or apoptosis, reference for instance being made to porin or members of the Bcl family as examples. Furthermore, a gene can be used, which codes for an expression product, which is for disease reasons not or to a small degree only generated. Finally, however not concludingly, the gene can code for iRNA, antisense nucleic acids, aptamers or ribozymes. It is understood that the transformation can be performed with several different genes. Suitably, but not necessarily, the gene for human applications will also be of human origin.

The invention comprises for instance the production of stem cells repopulating the hematopoietic system from neural stem cells by way of the treatment according to the invention and the use of such stem cells for producting pharmaceutical compositions for the treatment of diseases with pathologically reduced blood cell counts. The method can however of course also be used for other somatic stem cells, in order to produce cells repopulating the hematopoietic system or for the generation of cells, which are typical for other tissues/organs.

In all generality, the invention also comprises methods for treating diseases according to claim 8, wherein somatic stem cells are taken (preferably from the patient to be treated), these stem cells are subjected to a method according to claim 1, and the stem cells thus obtained having an increased development potential are galenically prepared to a pharmaceutical composition and administered to the patient.

Further, the invention also comprises pharmaceutical compositions containing stem cells having an increased development potential according to the invention.

Finally, the invention also comprises methods for finding substances, which are suitable for the treatment of somatic stem cells for producing stem cells having an increased development potential, wherein somatic stem cells are incubated with a potential substance or a mixture of such potential substances, wherein the incubated stem cells are subjected to the steps of Example 3, and wherein a substance or a mixture of substances is selected, if the administered incubated stem cells differentiate to more different tissues or cell types than with the same test steps, however without incubation of the somatic stem cells.

In the following, the invention is explained in more detail, based on figures representing an example of execution only. There are:

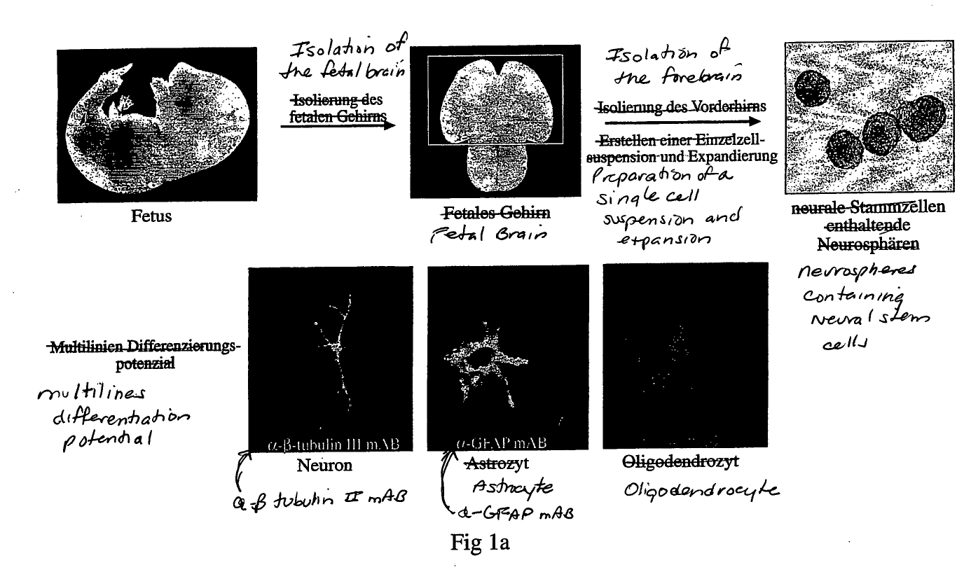

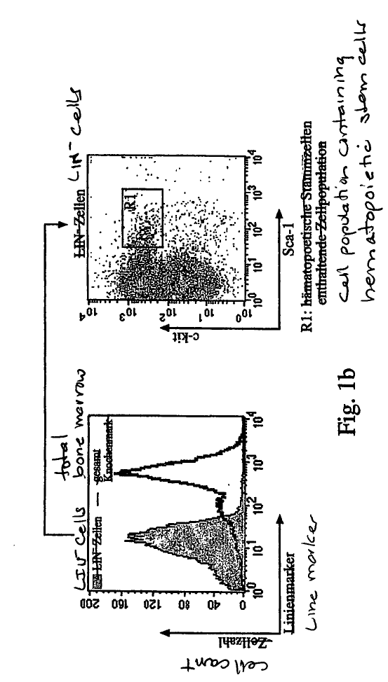

FIG. 1: the isolation of neural (a) and hematopoietic (b) stem cells of the mouse,

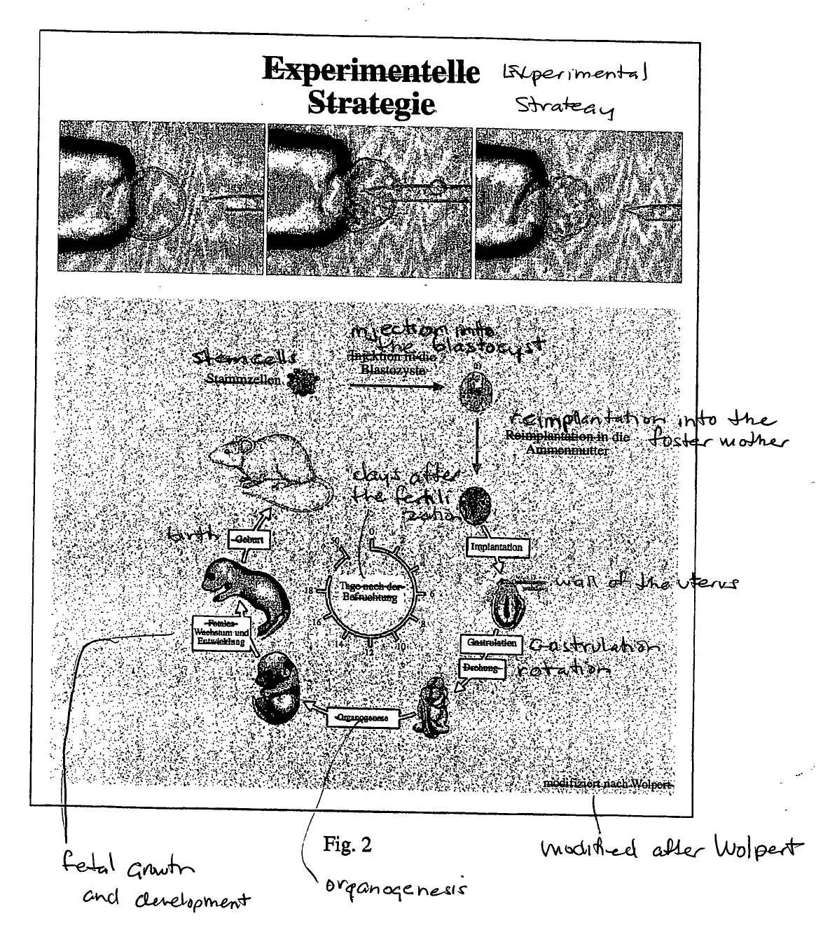

FIG. 2: the experimental strategy for the analysis of the development potential of somatic stem cells,

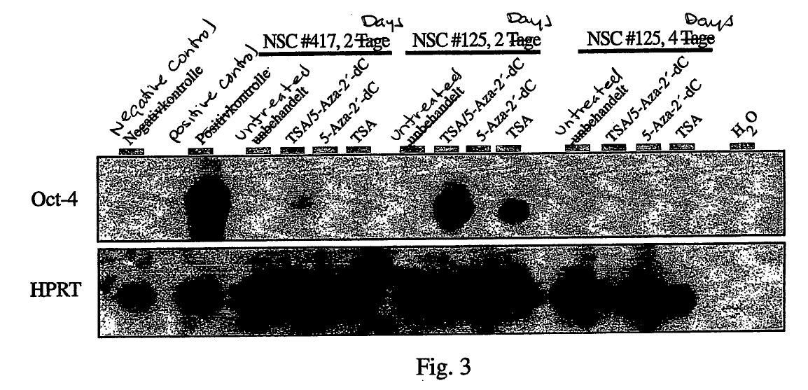

FIG. 3: the induction of gene expression of Oct4, as a marker of pluripotent gene expression after treatment with Trichostatin A and 5-aza-2′ deoxycytidine,

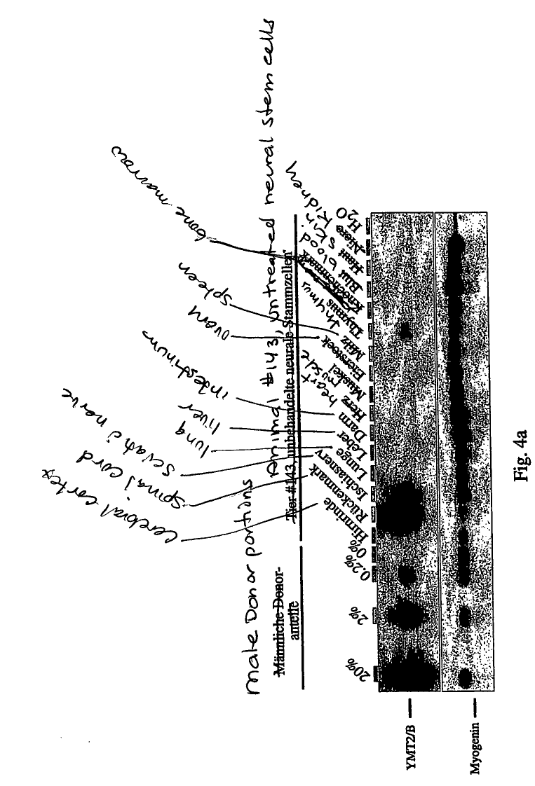

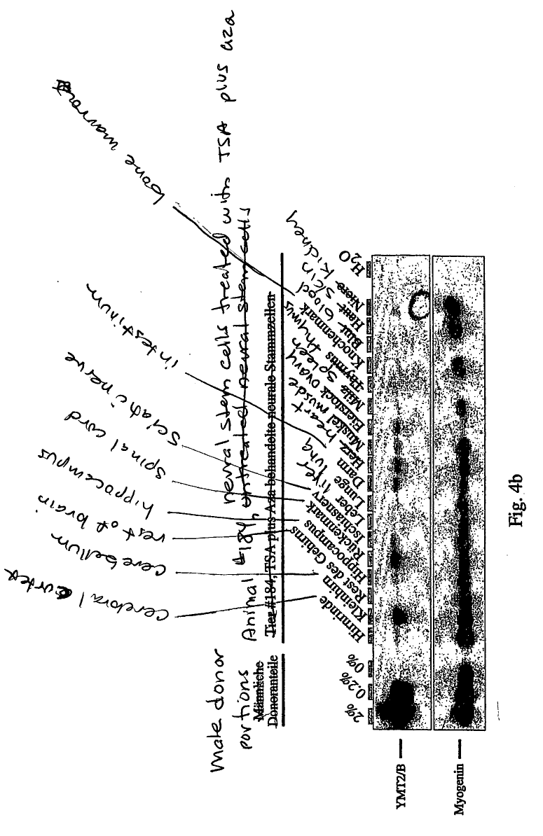

FIG. 4: the chimerism of adult mice after the injection of untreated (a) neural stem cells of the mouse or neural stem cells of the mouse treated with Trichostatin A (b) in blastocysts.

### EXAMPLE 1

### Isolation of Neural Stem Cells

For the isolation of neural stem cells, the forebrain was isolated from mouse fetuses or from the brain of adult animals and transferred into an single cell suspension. In presence of the neural growth factors EGF (epidermal growth factor) and FGF-2 (fibroblast growth factor 2), neural stem cells form little balls, so-called neurospheres. When the neurospheres are individualized, then from a cell a new neurosphere as well as neurons, astrocytes and oligodendrocytes may be generated after the division. The neural stem cells of FIG. 1a were then subjected to the method according to the invention.

### EXAMPLE 2

### Isolation of Hematopoietic Stem Cells

Hematopoietic stem cells were isolated from the bone marrow (BM) of adult mice through a negative/positive selection strategy by means of monoclonal antibodies. In a first step, all mature cells were depleted by antibodies, which are bound to small magnets. Non-bound cells (LIN⁻ cells) were dissociated by flow cytometry with two further antibodies, one of which is directed against the receptor tyrosine kinase c-kit and the other one against the stem cell antigen Sca-1. Hematopoietic stem cells of the mouse have the phenotype LIN⁻, c-kit⁺, Sca-1⁺. The results are shown in FIG. 1. The framed cell population of FIG. 1b was then subjected to the method according to the invention

### EXAMPLE 3

### Experimental Strategy for the Analysis of the Plasticity

In order to determine the plasticity of the employed somatic stem cell types, neural and hematopoietic stem cells of the Examples 1 and 2 were isolated and injected into mouse blastocysts (see FIG. 2). From the blastocyst develops the complete embryo and later the adult animal. By this method, the injected stem cells are subjected to all inductive processes, which take place during the development of the embryo. If the used hematopoietic or neural stem cells should have the capability to differentiate to all or many tissues or cell types of the adult animal, then descendants of the injected stem cells should be detectable for the developed animal in several different or all tissues and organs. If this detection is positive and the donor cells carry foreign, tissue-specific markers and perform foreign, tissue-specific functions, then the treatment of the somatic stem cells has led to an increased plasticity.

### EXAMPLE 4

### Treatment of Neural and Hematopoietic Stem Cells

For investigating whether the incubation of adult neural stem cells of Example 1 with deacetylase inhibitors (e.g. Trichostatin A) and/or nucleotide analogs (e.g. 5-aza-2′ deoxycytidine), which prevent a methylation, influences the gene expression, the stem cells were incubated with one of these substances or a mixture of these substances. This was performed in Neurobasal medium, B27 supplement, 20-40 ng EGF, 20-40 ng FGF-2, 150-250 nanomolar Trichostatin A and/or 300-600 nanomolar 5-aza-2′ deoxycytidine for 2 or 4 days. Subsequently to the incubation, RNA was isolated from the cells, and cDNA was produced by means of the enzyme reverse transcriptase. By using gene-specific primers, the gene expression of the Oct4 gene was investigated. The Oct4 gene serves as an example of a development-specific regulator gene. It is only active in very early development stages (zygote, morula, blastula). Later, the expression is restricted to germ cells. The Oct4 gene is not transcribed in the adult organism outside the germ cells, thus also not in somatic stem cells. The Oct4 gene is thus only active in cells, which have a development potential larger than that of the somatic stem cells [see Pesce M, Anastassiedies K, Scholer H R (1999) Oct4: lessons of totipotency from embryonic stem cells. Cells Tissues Organs 165:144-152]. Two different neural stem cell lines (NSC #417, NSC #125) according to Example 1 were either not treated (−/−), or incubated in Trichostatin A (TSA), 5-aza-2′ deoxycytidine (Aza) or in a combination of Trichostatin A and 5-aza-2′ deoxycytidine (+/+). The stem cells were treated for 2 or 4 days. FIG. 3 shows the results of the investigation of the induction of the gene expression by means of an Oct4-gene specific RT-PCR. For the normalization, a HPRT-specific RT-PCR was performed. As expected, the Oct4 gene is transcriptionally not active in untreated neural stem cells. 2-day incubation of neural stem cells with Trichostatin A or with a combination of Trichostatin A and 5-aza-2 deoxycytidine however reactivates the Oct4 gene. The combination of the two active ingredients shows additive effects with regard to the Oct4 expression. A transient effect of the treatment shows the 4-day incubation. Under these conditions, no Oct4 expression was detected anymore. This means in all generality that for the method according to the invention of an incubation with a substance reducing the methylation of the DNA and/or a substance promoting the chromatin acetylation, the duration of the incubation should be selected such that genes necessary for the increase of the plasticity, for instance Oct4, are activated.

Corresponding tests having identical results, which are not shown here, were performed with hematopoietic somatic stem cells. Herein, the incubation was made in DMEM medium, 20% FCS, IL3 (10 ng/ml), IL6 (20 ng/ml), SCF (50 ng/ml), 150-250 nanomolar Trichostatin A and/or 300-600 nanomolar 5-aza-2′ deoxycytidine for 2 or 4 days.

### EXAMPLE 5

### Increase of the Differentiation Properties

In order to investigate the development potential of somatic stem cells treated according to Example 4, neural stem cells of Example 4 (from male animals) were washed and injected (20-40 pieces) into blastocysts, which were isolated from superovulated females after object pairing. From the injected and retransfected blastocysts, normal and chimeric animals developed after transfer into foster mothers, and the female animals were examined in the age of 4 weeks with regard to the male donor portions in different tissues and organs. For this purpose, the animals were killed, and various tissues were isolated therefrom, and used for the preparation of genomic DNA. Male donor cells, which originate from the injected neural stem cells (NSC), were detected by a Y chromosome-specific PCR reaction (YMT primer). The result of a Southern Blot analysis is shown. The myogenin PCR serves as a control for the amount and quality of the employed genomic DNA. In FIG. 4a can be seen that untreated neural stem cells have mainly settled on neural tissues. From FIG. 4b can however be taken that stem cells treated with Trichostatin A have a substantially wider distribution spectrum. In none of the eight investigated animals, untreated neural stem cells participate in the generation of the bone marrow (BM) or the intestinum (gut). Both tissues settle however in four out of seven investigated animals after the injection of neural stem cells treated according to the invention.

In Table 1 are shown the results after the injection of murine hematopoietic stem cells (mHSC), untreated neural stem cells (mNSC) or treated neural stem cells (mNSC*). In bold letters are marked the tissues, which show an increased settlement. Abbreviations: brain: brain; cort.: cortex; cereb.: cerebellum; rest: remaining brain tissue; hip.: hippocampus; sp.c.: spinal cord; isch.: sciatic nerve; skin: skin; liv.: liver; heart: heart; musc.: muscle; spl.: spleen; thy.: thymus; BM: bone marrow; p.bl.: peripheral blood; kid.: kidney; lu.: lung; gut: intestinum; ov.: ovary.

### EXAMPLE 6

### Reconstitution of the Hematopoietic System by Means of Stem Cells According to the Invention

In order to immediately investigate whether neural stem cells treated according to the invention can generate hematopoietic cells, neural stem cells (four individual cell lines from male eGFP and Bcl-1 transgenic CD45.2 animals, treated and untreated) were transplanted by IV injection into sublethally irradiated adult female CD45.1 mice. A FACS analysis of the peripheral blood made 2.5 to 5 months after the transplantation for 11 animals, which were treated with untreated stem cells, showed that no hematopoietic top grafting had taken place. Rather, 5 out of 20 animals, which were treated with stem cells incubated with Trichostatin A and with 5-aza-2′ deoxycytidine, showed cells derived from the neural stem cells in the peripheral blood, which were eGFP+ and stained with the marker CD45.2. The hematopoietic chimerism in the peripheral blood was in the range from 5 to 65%. Donor-specific PCR at genomic DNA, which was isolated from the peripheral blood, confirmed the origin of the donor. Repeated investigations of the peripheral blood of an animal with 60% blood chimerism showed that the top grafting was stable over 5 months. Further analysis of splenocytes and bone marrow cells of all positive animals four months after the transplantation showed the presence of eGFP+cells derived from the neural stem cells, said eGFP+cells also staining with monoclonal antibodies against CD3 (T cells), CD19 (B cells) or Mac1 (macrophages). Furthermore, by a transplantation of bone marrow cells from primary receivers of stem cells according to the invention into secondary receivers, it was found that hematopoietic activity derived from the neural stem cells can serially be transplanted.

TABLE 1

| Tissue type | Tissue | mHCSs | mNSCs | mNSCs* |
|---|---|---|---|---|
| ectodermic | brain/cort. | 0/14 | 3/11 | 1/7 |
| | cereb. | | 0/7 | 1/7 |
| | rest. | | 0/7 | 0/7 |
| | hip. | | 3/10 | 4/7 |
| | sp.c. | 4/13 | 7/11 | 4/7 |
| | isch. | | 4/11 | 2/7 |
| | skin | 1/13 | 1/8 | 2/7 |
| mesodermic | liv. | 1/14 | 1/11 | 0/7 |
| | heart | 2/14 | 1/11 | 2/7 |
| | musc. | 1/14 | 3/11 | 2/7 |
| | spl. | 2/14 | 0/11 | 1/7 |
| | thy. | 6/14 | 1/8 | 2/7 |
| | BM | 4/14 | 0/8 | 4/7 |
| | p.bl. | 2/14 | 1/11 | 2/7 |
| | kid. | 0/5 | 0/11 | 1/7 |
| endodermic | li. | 2/14 | 1/11 | 4/7 |
| | gut | 1/14 | 0/8 | 4/7 |
| | ov. | 3/14 | 0/8 | 2/7 |

FIG. 1a

| | |
|---|---|
| Isolation of the | Isolation of the forebrain |
| fetal brain | Preparation of a single cell |
| | suspension and expansion |

| | | |
|---|---|---|
| Fetus | Fetal brain | Neurospheres containing |
| | | neural stem cells |

Multilines differentiation
potential

| α-β tubulin II mAB | α-GFAP mAB | |
|---|---|---|
| Neuron | Astrocyte | Oligodendrocyte |

FIG. 1b

LIN⁻ cells

| | |
|---|---|
| LIN⁻ cells | total |
| | bone marrow |

| Cell count | | c-kit |
|---|---|---|
| | Line marker | Sca-1 |
| | | R1: cell population containing |

hematopoietic stem cells

FIG. 2

Experimental

strategy

| | |
|---|---|
| stem cells | injection into the |

| | |
|---|---|
| blastocyst | reimplantation into the |
| | foster mother |

| | |
|---|---|
| birth | implantation |

| | |
|---|---|
| days after the | wall of the |
| fertilization | uterus |

| | |
|---|---|
| fetal growth | gastrulation |
| and development | |

rotation

organogenesis

modified after Wolpert

```
FIG. 3
negative control
  positive control
    untreated
      TSA/5-aza-2′-dC  NSC#417, 2 days
        5-aza-2′-dC
          TSA
            untreated
              TSA/5-aza-2′-dC  NSC#125, 2 days
                5-aza-2′-dC
                  TSA
                    untreated
```

```
TSA/5-aza-2′-dC NSC#125, 4 days
  5-aza-2′-dC
    TSA
```

Oct-4 H₂O
HPRT

FIG. 4a
Male donor

| | |
|---|---|
| portions | Animal #143, untreated neural stem cells |

```
20%
  2%
    0.2%
      0.0%
        cerebral cortex
          spinal cord
            sciatic nerve
              lung
                liver
                  intestinum
                    heart
                      muscle
                        ovary
                          spleen
                            thymus
                              bone marrow
                                blood
YMT2/B  skin
                                    kidney
Myogenin  H₂O
FIG. 4b
Male donor
```

| | |
|---|---|
| portions | Animal #184, neural stem cells treated with |
| | TSA plus aza |

```
2%
  0.2%
    0%
      cerebral cortex
        cerebellum
          rest of brain
            hippocampus
              spinal cord
                sciatic nerve
                  liver
                    lung
                      intestinum
                        heart
                          muscle
                            ovary
                              spleen
                                  thymus
                                    bone marrow
                                      blood
YMT2/B  skin
                                          kidney
Myogenin H₂O
```

In the claims:

1. A method for producing stem cells having an increased development potential from somatic stem cells,
wherein a tissue sample comprising somatic stem cells or a body fluid sample comprising somatic stem cells is taken from an organism,
wherein from this tissue sample or body fluid sample somatic stem cells are isolated and/or cultivated and/or transformed with a defined foreign nucleic acid, which is under the control of an operatively connected regulatory element, and
wherein the somatic stem cells are treated with a substance modulating the methylation of the DNA of the cells or with a substance modulating the acetylation of chromatin of the cells.

2. A method according to claim 1, wherein the substance modulating the methylation of the DNA of the cells is selected from the group comprising methylation inhibitors, not methylatable nucleotide analogs, methylase inhibitors, demethylase activators, mixtures of such substances, and the antagonists thereof.

3. A method according to claim 1 or 2, wherein the substance modulating the chromatin acetylation is selected from the group comprising acetylation activators, histone acetylase activators, histone deacetylase inhibitors, mixtures of such substances, and the antagonists thereof.

4. A method according to one of claims 1 or 2, wherein the somatic stem cells comprise neural or hematopoietic stem cells.

5. Somatic stem cells having an increased development potential, obtainable by the steps comprising:
taking from an organism a tissue sample comprising somatic stem cells or a body fluid sample comprising somatic stem cells;
isolating, cultivating or transforming the somatic stem cells from this tissue sample or body fluid sample with a defined foreign nucleic acid, which is under the control of an operatively connected regulatory element; and
treating the obtained somatic stem cells with a substance modulating the methylation of the DNA of the cells or a substance modulating the acetylation of chromatin of the cells.

6. A pharmaceutical composition prepared from the somatic stem cells of claim 5.

7. The composition of claim 6, wherein the stem cells are autologous.

8. The composition of claim 6 or 7 wherein the composition is used to treat degenerative diseases of the central nervous system, Parkinson's disease, diabetes, diseases with pathologically reduced blood cell counts, muscular dystrophy, HSC transplantation after high-dose chemotherapy/radiotherapy for cancer therapies; myocardium cell replacement after a heart attack, skin replacement, cartilage replacement, liver regeneration after cirrhosis of liver, metabolic diseases or age-related tissue degeneration.

9. A pharmaceutical composition containing stem cells according to claim 5 further comprising a mixture with galenic auxiliary or carrier substances or at least one therapeutically effective substance.

10. A method according to claim 3, wherein the somatic stem cells comprise neural or hematopoietic stem cells.
